(19) United States
(12) Patent Application Publication
(10) Pub. No.: US 2012/0157875 A1
(43) Pub. Date: Jun. 21, 2012

(54) METHODS OF DIAGNOSIS AND OF SCREENING FOR ELECTRICAL MARKERS FOR HIDDEN (OCCULT) MALADIES AND MODULATION OF ENDOGENOUS BIOELECTRICAL NEURONAL SIGNALS IN PATIENTS

(75) Inventors: Michel Afargan, Raanana (IL); Elia Bernardino Ricci, Cascinette d'Ivrea (IT)

(73) Assignee: LIFEWAVE LTD., Petach Tivka (IL)

(21) Appl. No.: 13/388,991

(22) PCT Filed: Aug. 4, 2010
(86) PCT No.: PCT/IB10/53531

§ 371 Date: Feb. 28, 2012

Related U.S. Application Data
(63) Continuation-in-part of application No. PCT/IB09/54708, filed on Oct. 25, 2009.
(60) Provisional application No. 61/231,035, filed on Aug. 4, 2009.
(60) Provisional application No. 61/231,036, filed on Aug. 4, 2009.

(51) Int. Cl. A61B 5/05; A61N 1/00

(52) U.S. Cl. 600/547; 607/68

(57) ABSTRACT

A method for diagnosing non-visible (occult) maladies in a human patient, the method comprising: (a) deploying at least two electrodes spaced apart on the skin of the patient; (b) detecting and recording a bioelectrical signal in and around said electrodes, the bioelectrical signal being a stochastic signal; (c) transforming the stochastic signal into a voltage versus frequency spectra using a Fast Fourier Transform (FFT) algorithm; (d) comparing a graph of a resultant FFT level of the patient to at least one graph of a baseline FFT level; and (e) determining a presents of a non-visible (occult) malady based on said comparison. Methods for monitoring a treatment regimen for non-visible (occult) maladies and for modulating the amplitude of endogenous bioelectrical stochastic signals in a human patient are also disclosed.

### FIELD AND BACKGROUND OF THE INVENTION

The present invention relates to a method for diagnosis and prognosis of hidden (occult) damaged, or turned over, of animal tissues, including human tissue by detection of an endogenous bioelectric current flow through apparently healthy body tissue. The invention relates to a method and procedure for measuring, recording and analyzing the bioelectrical field in and around areas of a living body and in particular the method identifies and defines a discrete bioelectrical profile of specifically hidden (occult) maladies. Also included is a method for modulation of the endogenous bioelectrical signals in humans.

Electrophysiology is the science and branch of physiology that delves into the flow of ions in biological tissues, the bioelectrical recording techniques which enable the measurement of this flow and their related potential changes. One system for such a flow of ions is the Power Lab System by ADInstruments headquartered in Sydney, Australia. Another system is the LifeWave™ BST, from LifeWave Hi-Tech Medical Devices Ltd. of Petach Tikva, Israel, the present assignee. The LifeWave™ BST can be also be used as a diagnostic device. U.S. Pat. Nos. 6,363,284, 6,393,326 and 6,941,173, to the present assignee and are hereby incorporated into this disclosure in their entirety as if fully set forth herein, related to the BST device, the bi-polar wave form it generates and methods for treating sores. The endogenous bioelectrical stochastic signals discussed herein were first described in U.S. Pat. Nos. 6,363,284, 6,393,326 and 6,941,173, however, the full extent of their use was not fully understood at that time.

Clinical applications of extracellular recording include among others, the electroencephalogram and the electrocardiogram. To understand these biomedical signals, it is necessary to understand signal types, properties and statistics.

Deterministic signals are exactly predictable for the time span of interest. Deterministic signals can be described by mathematical models.

Stochastic or random signals are those signals whose value has some element of chance associated with it, therefore it cannot be predicted exactly. Consequently, statistical properties and probabilities must be used to describe stochastic signals. In practice, bioelectrical signals often have both deterministic and stochastic components.

Regarding signal amplitude statistics, a number of statistics may be used as a measure of the location or “centre” of a random signal. These include,
The mean, which is the average amplitude of the signal over time.
The median, which is the value at which half of the observations in the sample have values smaller than the median and half have values larger than the median. The median is often used as the measure of the “centre” of a signal because it is less sensitive to outliers.
The mode, which is the most frequently occurring value of the signal.
The maximal and minimal amplitude, which are the maximal and minimal value of the signal during a given time interval.
The range or peak-to-peak amplitude, which is the difference between the minimum and maximum values of a signal.

Regarding continuous time signals versus discrete time signals, signals are continuous time signals when the independent variable is continuous; therefore the signals are defined for a continuum of values of the independent variable X(t). An analogue signal is a continuous time signal. Discrete time signals are only defined at discrete times; the independent variable takes on only a discrete set of values X(n). A digital signal is a discrete time signal.

A discrete time signal may represent a phenomenon for which the independent variable is inherently discrete (e.g., amount of calories per day on a diet). On the other hand, a discrete signal may represent successive samples of an underlying phenomenon for which the independent variable is continuous (e.g., a visual image captured by a digital camera is made of individual pixels that can assume different colors).

There are quantitative methods to measure the frequency and amplitude of a waveform. One of the most well known is called spectral analysis: any waveform can be mathematically decomposed in a sum of different waveforms. This is what the so-called Fourier analysis does; it decomposes the waveform in different components and measures the amplitude (power) of each frequency component. What is plotted is a graph of power (amplitude) vs. frequency.

Whereas research on direct current (DC) activity in wound healing and tissue remodeling has a long history, bioelectric fields of alternating current (AC) with specific frequencies have been much less studied.

Specific frequencies have been detected in various biological pathways known to be associated with wound healing such as pain, cell metabolism, inter-cellular communication and bone growth. However, due to the absence of suitable measurement tools, there has been no definitive proof of involvement of AC with defined frequency spectra in tissue injuries or bleeding.

While performing research relating to a diagnostic method that identifies and defines a discrete bioelectrical profile of a wound during a healing, worsening or stopped condition so as to provide a prognosis for such wounds as disclosed in PCT/IB09/54708, the present inventors noticed certain anomalies in the control group of subjects with no known wound or injury. This lead to the discovery that two of the female members of the control group were experiencing their period at the time their bioelectrical profiles were being taken. A comparison of the bioelectrical profiles of these two subjects showed them to be similar to each other and distinct from the rest of the control group, just as were the subjects of the study that had wounds. Therefore, the bioelectrical profiles of the two females having their period provided a discrete bioelectrical profile of hidden (occult) bleeding.

Based on this discovery, the present inventors performed further studies relating to non-visible (occult) type maladies including Central Nervous System (herein CNS) maladies such as Alzheimer dementia, stroke and spinal cord injuries which lead to the understanding that the bioelectrical signals they were identifying were endogenous stochastic signals.

Further, to date no diagnostic method based on a discrete bioelectrical profile for non-visible wound related maladies, such as occult bleeding, or for CNS anomalies has been ventured in the medical field.

There is therefore a need for a diagnostic method that identifies and defines a discrete bioelectrical profile of hidden (occult) maladies using a non invasive manner so as to provide a prognosis for such conditions. It would be beneficial if there was also included is a method for modulation of the endogenous bioelectrical signals in humans.

### SUMMARY OF THE INVENTION

The present invention is a diagnostic method that identifies and defines a discrete bioelectrical profile of hidden (occult) maladies so as to provide a prognosis for such condition and a method for modulation of the endogenous bioelectrical signals in humans.

According to the teachings of the present invention there is provided, a method for diagnosing non-visible (occult) maladies in a human patient, the method comprising: (a) deploying at least two electrodes spaced apart on the skin of the patient; (b) detecting and recording a bioelectrical signal in and around the electrodes; (c) transforming the bioelectrical signal into a graph; (d) comparing the resultant graph of the patient to at least one graph of a baseline of normal healthy humans; and (e) determining a presents of a non-visible (occult) malady based on the comparison.

According to a further teaching of the present invention, the deploying of the electrodes in on an area of a leg of the patient.

According to a further teaching of the present invention, the detecting and recording a bioelectrical signal is implemented as detecting and recording a stochastic signal.

According to a further teaching of the present invention, steps 1(c) and 1(d) are implemented as: (a) transforming the stochastic signal into a voltage versus frequency spectra using a Fast Furier Transform (FFT) algorithm; and (b) comparing a graph of a resultant PET level of the patient to at least one graph of a baseline FFT level of normal healthy humans.

There is also provided according to the teachings of the present invention, a method for monitoring a treatment regimen for non-visible (occult) maladies in a human patient, the method comprising: (a) deploying at least two electrodes spaced apart on the skin of the patient; (b) detecting and recording a first bioelectrical signal in and around the electrodes, the bioelectrical signal being a first stochastic signal; (c) transforming the first stochastic signal into a first voltage versus frequency spectra using a Fast Furier Transform (FFT) algorithm; (d) establishing a graph of a resultant FFT level as a baseline FFT level for the patient; (e) administering the treatment regimen; (f) redeploying the electrodes after a predetermined passage of time; (g) detecting and recording at least a second bioelectrical signal in and around the electrodes, the bioelectrical signal being a second stochastic signal; (h) transforming the second stochastic signal into a second voltage versus frequency spectra using a Fast Furier Transform (FFT) algorithm; (i) comparing a graph of a resultant FFT level of the patient during treatment to the graph of the baseline FFT level for the patient; and (j) determining success of the treatment regimen based on the comparison.

According to a further teaching of the present invention, steps 6(f)-6(j) are repeated according to a predetermined time table.

There is also provided according to the teachings of the present invention, a method for modulating the amplitude of endogenous bioelectrical stochastic signals of a human, the method comprising: (a) deploying at least two spaced-apart electrodes in contact with a skin surface of the human; (b) externally inducing a percutaneous flow of bioelectrical stochastic signals between the electrodes; wherein the bioelectrical stochastic signals have a bipolar voltage wave form that substantially mimics a bipolar voltage wave form produced by a human body.

According to a further teaching of the present invention, there is also provided increasing the amplitude of the endogenous bioelectrical stochastic signals by implementing steps 7(a) and 7(b).

### BRIEF DESCRIPTION OF THE DRAWINGS

The invention is herein described, by way of example only, with reference to the accompanying drawings, wherein:

FIG. 1 illustrates the placement of electrodes on a healthy limb;

FIG. 2 is a graph of the FFT level baseline for healthy subjects;

FIG. 3 is a graph of the FFT level baseline for healthy subjects and the FFT level for hidden bleeding of two women during period;

FIG. 4 is a graph of the FFT level baseline for healthy subjects, the FFT level for subjects with chronic wounds (without CNS anomalies) and the FFT level for subjects with chronic wounds but with diagnosed central neurological diseases;

FIG. 5 is a graph of the FFT level baseline for healthy subjects, the FFT level of patients with Alzheimer dementia and the FFT level of patients with stroke;

FIG. 6A is a graph of the FFT level measured on the arms of subjects with spinal cord injury;

FIG. 6B is a graph of the FFT level measured on the legs of the same subjects with spinal cord injury;

FIG. 7A is the graph of the FFT levels for a first patient with Multiple sclerosis and having a chronic wound measured near the wound;

FIG. 7B is the graph of the FFT levels for the patient of FIG. 7A measured on the contralateral healthy limb;

FIG. 8A is the graph of the FFT levels for a second patient with Multiple sclerosis and having a chronic wound measured near the wound;

FIG. 8B is the graph of the FFT levels for the patient of FIG. 8A measured on the contralateral healthy limb;

FIG. 9A is the graph of the FFT levels for a first patient having suffered a stroke and having a chronic wound measured near the wound;

FIG. 9B is the graph of the FFT levels for the patient of FIG. 9A measured on the contralateral healthy limb;

FIG. 10A is the graph of the FFT levels for a second patient having suffered a stroke and having a chronic wound measured near the wound;

FIG. 10B is the graph of the FFT levels for the patient of FIG. 10A measured on the contralateral healthy limb;

FIG. 11A is the graph of the FFT levels for a patient with diabetic neuropathy and having a chronic wound measured near the wound;

FIG. 11B is the graph of the FFT levels for the patient of FIG. 11A measured on the contralateral healthy limb;

FIG. 12 is the graph of the FFT levels for the patient before and after administration of general anesthesia before surgery;

FIG. 13 is the graph of the FFT levels for the patient after administration of general anesthesia before and during surgery;

FIG. 14 is the graph of the FFT levels for the patient after administration of spinal anesthesia before and during surgery; and

FIG. 15 is the graph of the FFT levels for the patient after administration of local anesthesia before and during surgery.

### DESCRIPTION OF THE PREFERRED EMBODIMENTS

The present invention relates to a diagnostic method that identifies and defines a discrete bioelectrical profile of hidden (occult) maladies and a method for modulation of the endogenous bioelectrical signals in humans.

The principles and operation of a diagnostic method that identifies and defines a discrete bioelectrical profile of hidden (occult) maladies according to the present invention may be better understood with reference to the drawings and the accompanying description.

By way of introduction, bioelectrical flow in the body plays a major role in many physiological and pathophysiological conditions. During tissue injury associated with bleeding, direct bioelectrical current known as “the current of injury” is triggered (or generated) around the wound. Endogenous alternating current (AC) or stochastic (random) currents that characterized by specific frequencies are mainly attributed in medicine to the action of nerves.

As mentioned above, the endogenous bioelectrical stochastic signals discussed herein were first described in U.S. Pat. Nos. 6,363,284, 6,393,326 and 6,941,173, however, the full extent of their use was not fully understood at that time. Therefore, the present inventors and colleagues used the knowledge base obtained during the development and testing of the LifeWave™ BST as the starting point of their research relating to a diagnostic method that identifies and defines a discrete bioelectrical profile of a wound during a healing, worsening or stopped condition so as to provide a prognosis for such wounds as disclosed in PCT/IB09/54708 identified in humans the presents of discrete alternating current signals that are specific to patients with chronic wounds and acute wounds in comparison to healthy subjects. They conducted simultaneous alternating current measurements on the same patients at their injury where there was an existing wound with bleeding and on the contralateral non injured limb. They then activated an algorithm to transform these stochastic signals to frequency spectra and found that the same signal pattern exists around the wound and on the contralateral non-injured side. These discrete microcurrent signals display unique frequency profiles within the range of 0.1-1000 Hz (amplitude range of nano to micro volts). Furthermore, bioelectrical recordings of wounds taken during an acute injury state induced by debridement of chronic wounds revealed an instantaneous stochastic signal with a frequency pattern exceeding 1000 Hz, a signal that was triggered simultaneously around the acute wound and on the contralateral healthy limb of the same patient.

These findings indicate that stochastic resonance may be associated with the wound healing process. As mentioned above, these stochastic signals were first described in U.S. Pat. Nos. 6,363,284, 6,393,326 and 6,941,173. These patents relate to the LifeWave™ BST device and the bipolar voltage wave form it generates. This bipolar voltage wave described and claimed there as substantially mimicking a bipolar voltage wave form produced by a human body.

From a neurophysiological and therapeutic standpoint, this work suggests that stochastic signals associated with wounds may be linked to a neurological “cross talk” between the wound and the nervous system and may serve as a target for wound therapy.

Whereas research on direct current (DC) in wound healing and tissue remodeling has a long history, bioelectric fields of alternating current (AC) and stochastic current with specific frequencies have been much less studied.

Specific frequencies have been detected in various biological pathways known to be associated with wound healing such as pain, cell metabolism, inter-cellular communication and bone growth. However, due to the absence of suitable measurement tools, there has been no definitive proof of involvement of stochastic bioelectrical signals with defined frequency spectra in wounds.

There is accumulating evidence that sensory nerves may play an important role in tissue repair. While most of these studies have been done on animals, the effect of sensory nerve activation in human wound healing remains mostly unexplored.

While intuitively, noise should impede signal detection, a wide range of studies from computer models to human experiments has demonstrated that low-level mechanical or electrical noise presented directly to sensory neurons can significantly enhance their ability to detect weak stimuli. This phenomenon of noise improving sensory performance is termed stochastic resonance.

It has been shown that localized electrical stimulation of noise to the lower extremities of elderly adults may improve postural control and tactile sensitivity throughout the stimulation of sensory nerves. Stochastic resonance enhances sensation in patients with diabetic neuropathy and may affect tissue repair on the molecular and cellular level.

We recently reported on a proof-of-principle study using stochastic electrical stimulation to treat hard-to-heal wounds (wounds that were resistant to standard, advanced and even to very intricate treatments for years). The treatment was applied by the BST (Bioelectrical Signal Therapy) device which transmits stochastic electrical noise (“white” noise) with most of power from 0 to 1000 Hz and a current density of 0.3 mA/cm2. Following 60 consecutive days of treatment the wounds surface area was reduced by an overall mean closure rate of 82.5% (SD=25.2%). This open-label observational case series was the first indication of the possible role of stochastic resonance in wound healing.

The objective of the research was to elucidate whether oscillating characteristics of specific frequency components exist around injured tissues in humans. They wished to identify discrete stochastic cues linked to a specific spectrum of frequencies adjacent to chronic non-healing wounds and to determine whether these stochastic cues are specific to this group of patients.

For this objective, on the same group of patients the researchers conducted bioelectrical recordings on both injured and on non-injured tissue, with the measurements on non-injured tissue to be used as control data.

For bioelectrical recordings the researchers affixed two electrodes on both proximal and distal sides across the medial axis of the injured skin and signals were measured against the third ground electrode. In order to amplify the specificities of the recorded stochastic signals a Fast Fourier Transform (FFT) algorithm was used. By this signal processing approach they were able to profile discrete signals with significant differences in amplitude (voltage) and/or frequency within a filter set at 0.1 to 1000 Hz.

To establish the baseline levels of the bioelectrical measurements, a group of healthy subjects (no wounds) was recruited and the graph of their mean FFT levels served as the minimal amplitude levels (i.e., baseline levels).

To test the role of endogenous electrical frequencies in damaged tissue, the inventors conducted electrical measurements on patients with chronic wounds as target population. Chronic wounds are trapped in a non-advancing phase of healing and are unable to progress through the sequential stages of tissue repair. Compared to acute healing wounds, studies have been shown that human chronic wounds differ in their biochemical, molecular and mechanistic characteristics such as reduced levels of metalloproteinase inhibitors and diminished growth factor activity. Therefore, unlike acute wounds that are dynamically changed in time, chronic wounds may be considered relatively stable and thus could provide an example of the profile of their mean electric fields. The mean electrical measurements around chronic wounds exhibited significantly higher amplitude (voltage) above the baseline measurements in healthy subjects. These stochastic signals were characterized by mean electrical frequency spectra within the range of 0.1 to 1000 Hz. The mean maximum voltage (Vmax) of this signal was found in the range of 0.1 to 50 Hz (a frequency range considered as environmental electrical radiation). The signal reduced exponentially to its minimal voltage (Vmin) of about 7 nV which was detected around 1000 Hz. Due to the significantly weak/absence amplitude of such signals in the baseline group of healthy subjects we confirmed that this discrete signal is specific to chronic wounds.

In order to confirm that the specific signal detected around wounds is specific to the wound site, the inventors conducted simultaneously the same measurement on the contralateral healthy limb of the same patients. Intriguingly, they found in the same patients that the stochastic waveform that exists around wounds, overlapped with the same electrical frequency spectra and amplitude of the signals recorded on the contralateral non-injured organ. The inventors deduced that the discrete stochastic signals found in patients with chronic wounds could also serve as a systemic parameter in the body. These statistically significant results highlight the possibility that chronic wounds may be studied as local tissue damage with systemic attributes.

Furthermore, the preliminary electrical recordings on anesthetized patients (blockage of sensory nerves) show that during incision i.e., an acute wounding, the inventors detected stochastic signals with considerably weak amplitudes (around the baseline levels), another indication that nerves or nerve injury may be involved in the stochastic signaling during acute injury.

The existence of defined specific electrical frequencies in the central nervous system is well documented in medicine, and these are fundamental markers in the monitoring and studies of brain activity. Despite studies on pain, the role of electrical frequencies in other peripheral disorders such as tissue injury has been much less studied.

The finding of the existence of systemic signals in chronic wound patients provides a new insight on the pathophysiology of wound healing on the systemic level. With regard toward clinical practice, both laboratory and clinical classification of chronic wounds is still an un-met need in wound care. The results suggest that electrical frequency spectra may be considered a potential neurophysiological descriptor for evaluating the processes that are likely to affect chronic wound healing and healing end points. The findings on the possible involvement of the nervous system in chronic and acute wounds should be further explored. Their studies elucidate electrical frequencies around tissue injuries that overlap with the body's signals.

Surprisingly, identified within the healthy subjects group several were samples with distinct discrete signals. Review of the records of these subjects revealed that these discrete signals are specific to healthy women during their period.

Based on this discovery, the present inventors performed further studies relating to non-visible (occult) type maladies including Central Nervous System (herein CNS) maladies such as Alzheimer dementia, stroke, spinal cord injuries, Multiple Sclerosis, and diabetic neuropathy.

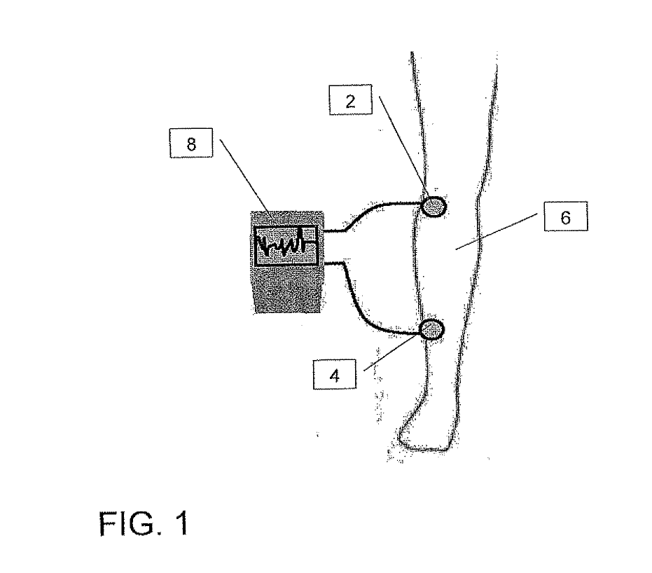

Turning now to the drawings, FIG. 1 illustrates the placement of the electrodes 2 and 4 on the leg 6 of the patient. The electrodes in turn are in bioelectrical communication with a device 8 for at least recording and preferably also filtering the electronic signal detected by the electrodes. It should be noted that although the leg is the preferred location for placement of the electrodes, the signals detected and used for the method of the present invention are systemic in nature and may be detected to some degree in substantially any area of the body.

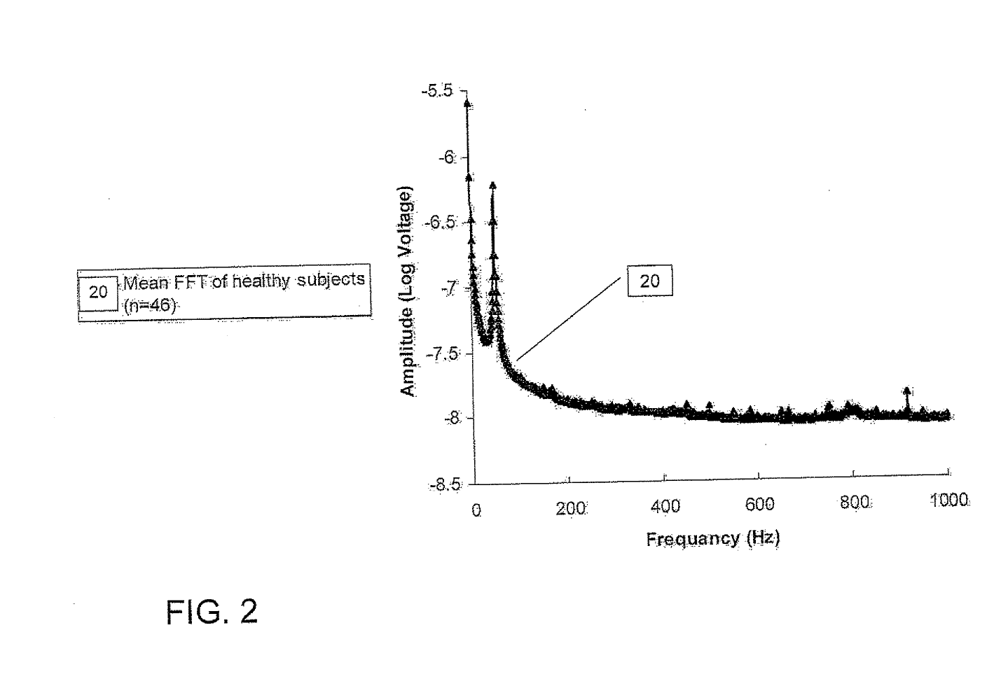

FIG. 2 is a graph of the Mean FFT level 20 of the healthy subjects in the control group. This graph is used as the baseline graph to which the FFT level graphs of the non-visible maladies are compared.

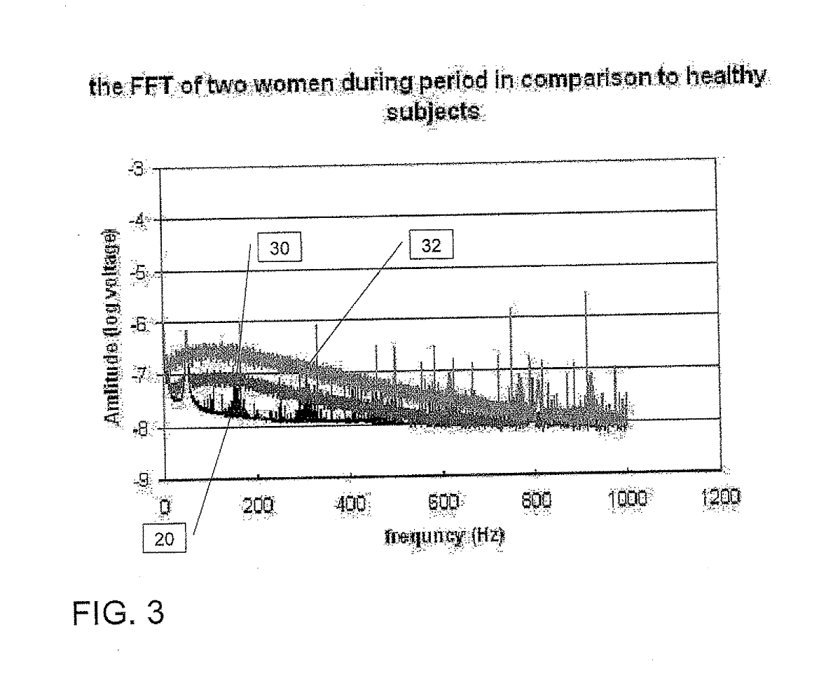

FIG. 3, is graph of the FFT levels 30 and 32 of two women with menstrual bleeding in comparison to the baseline FFT level 20 of the healthy subjects from the control group.

The signals are significantly different in comparison to the control group. However, it will be appreciated that while the amplitude to the two curves 30 and 32 differ, the shape of the curves is very similar and may be indicative of menstrual bleeding. This, therefore, provides the basis for a method for diagnosing hidden bleeding in apparently healthy individuals.

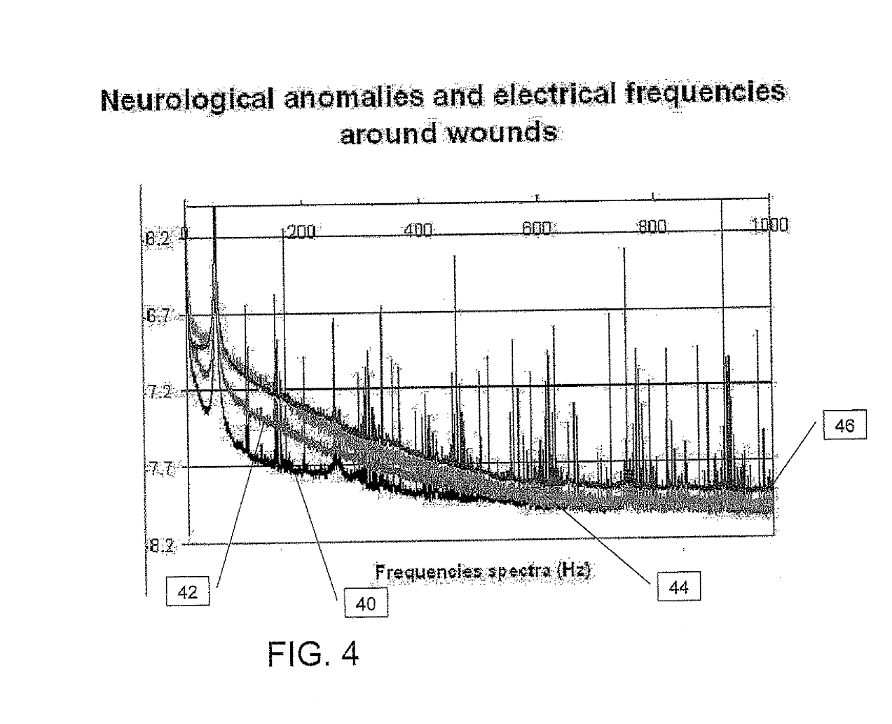

FIG. 4 provides some background from the research that lead to the present invention. The researchers started their research with wounds and wanted to show the interaction of the CNS with wounds. Shown here are the baseline mean FFT levels for subjects with chronic wounds and with CNS comorbidity with the measurement being taken around the wound (curve 40), the mean FFT levels for subjects with chronic wounds but with CNS comorbidity with the measurement being taken on the contra lateral limb (curve 42), the mean FFT level taken on healthy skin of patients with CNS anomalies but no wounds (curve 44), and the mean FFT levels for subjects with chronic wounds but with no CNS comorbidity with the measurement being taken around the wound (curve 46).

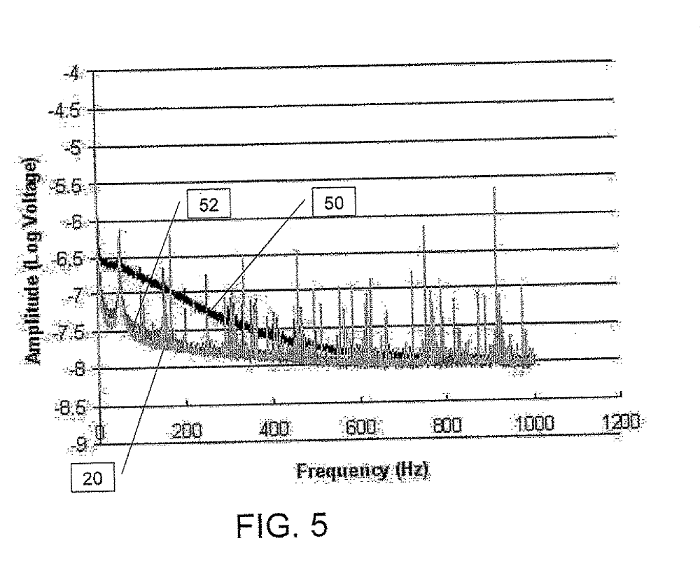

It will be readily appreciated that the subjects with chronic wounds and with CNS comorbidity possess a reduced FFT level 40 compared to the FFT level 46 with chronic wounds but with no CNS anomalies. This was the first indication regarding the role of the CNS in wounds. In the next step the inventors used subjects with CNS anomalies without wounds and found the novel differences within that group (Alzheimer in comparison to stroke) that are disclosed with regard to FIG. 5.

The graph of FIG. 5 shows the FFT levels of patients that do not have any wounds. This group was originally used as the control groups to those who had chronic wounds and CNS anomalies. It will be readily understood from this graph that patients with dementia (in these cases—Alzheimer Dementia—herein AD) show a significantly higher FFT level 50 in comparison to the FFT level 52 of patients with stroke. Both of which are different than the baseline FFT level 20 of the healthy subjects. The intriguing result is the case of AD.

The present inventors assert that they have identified an endogenous stochastic signal whose variance from an identified healthy baseline state is indicative of the state of wellbeing of a human body. The present inventors suggest that the measurement method on the present invention which is based on this endogenous stochastic signal may be used for (by non-limiting example):

1. Diagnosis and prognosis at early stages of neurodegenerative diseases that are known to be associated with ischemia of neurons such as, but not limited to, tissue damage within the brain, Alzheimer, Parkinson, stroke, multiple sclerosis, epilepsy, depression, ALS (although peripheral—yet a neurological damage), paraplegia and diabetic neuropathy; and

2. As a Marker for monitoring the effects of treatment, including drugs, on a patient both during and after the treatment regimen.

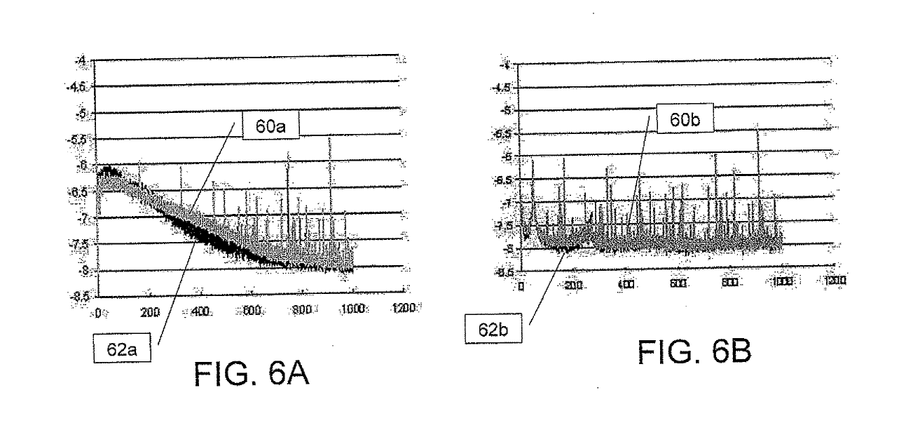

FIGS. 6A and 6B present graphs of the FFT levels of two subjects that had no visible wounds but had suffered spinal cord. The PET levels 60a and 62a shown in FIG. 6A were measured on the arms of the patient's, which were above the level of the spinal injury. The FFT levels 60b and 62b shown in FIG. 6B were measured on the patient's leg, which were below the level of the spinal injury.

It should be noted that the FFT levels 60a and 62a of FIG. 6A taken above the spinal injury are very high. In fact, these FFT levels were among the highest FFT levels found during the study. In contrast, the FFT levels 60b and 62b of FIG. 613 taken below the spinal injury are much lower by comparison, as was expected.

Based on the above research, the method of the present invention for detecting the prognosis of central and or peripheral neurological diseases as well as non-visible internal bleeding or other injury in a patient was developed. The method of present invention can also be used for monitoring the effects of various therapeutics on the prognosis of the malady being tracked according to a predetermined time table.

Such a method may be used to monitor the effectiveness of treatment by establishing a current baseline which could be used for comparison to later graphs generated at intervals during the treatment regimen to determine if the signals (graphs) are moving toward a “normal” curve, or otherwise indicative of change in the patient's condition.

As part of the study, once baseline data was established those patients with chronic wounds were treated and their progress was tracked. Treatment was conducted with a LifeWave™ BST (Bioelectrical Signal Therapy) device that was used according to the manufacturer's instructions.

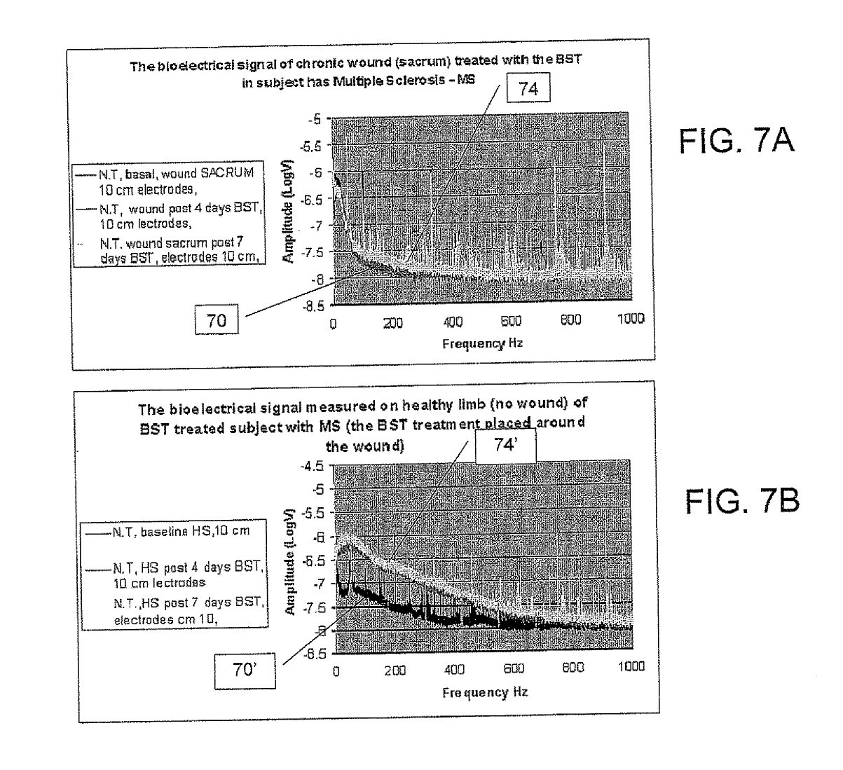

The graphs of FIGS. 7A-11B show the FFT levels for five patients that had chronic wounds with CNS comorbidity. While the graph of FIG. 4 shows the mean baseline bioelectrical signal measurements of the different groups, these graphs show the FFT levels for individual patients tracked during the duration of treatment to the chronic wound. Surprisingly, the inventors noticed an increase in FFT levels recorded on the contralateral limb by the end of the treatment.

Specifically, FIGS. 7A and 7B show the graphs of the FFT levels for a first patient with Multiple sclerosis and having a chronic wound. The graph of FIG. 7A shows little change in the FFT levels from the baseline FFT 70 to the FFT level 74 of day seven of treatment. However, the graph of FIG. 7B shows there is a marked increase in the FFT level on the contralateral healthy limb from the baseline FFT level 70′ to the FFT level 72′ of the fourth day of treatment and the further increase of the FFT level 74′ of the seventh day of treatment.

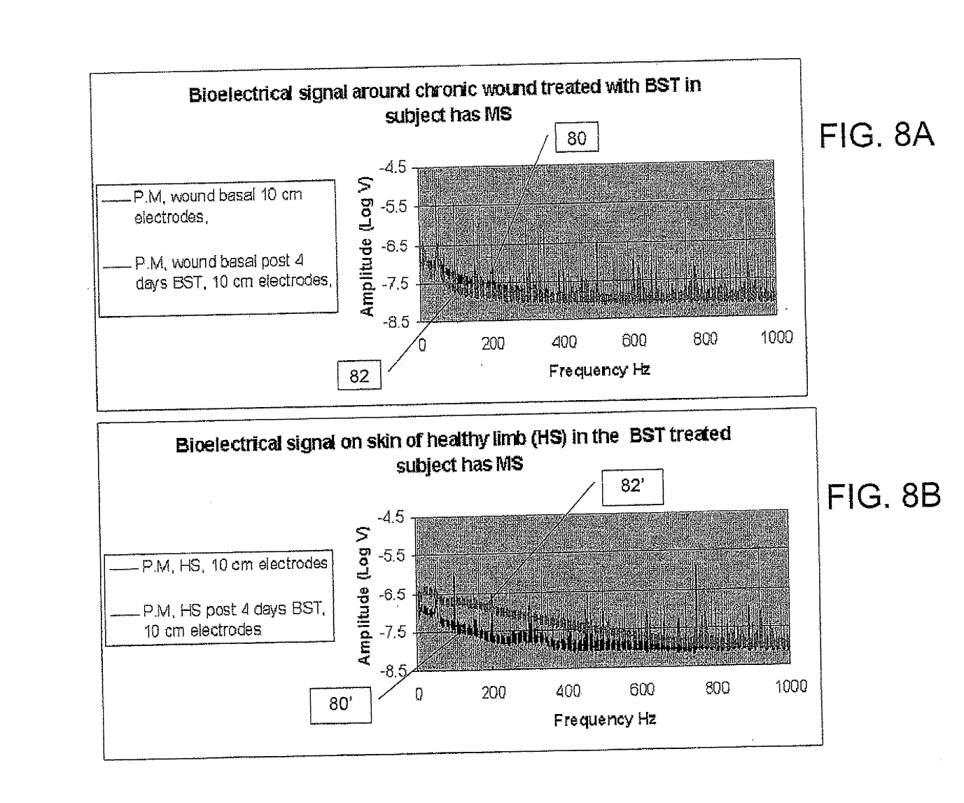

FIGS. 8A and 8B are the graphs of the FFT levels for a second patient with Multiple sclerosis and having a chronic wound. Here too, the graph of FIG. 8A shows little change in the FFT levels from the baseline FFT 80 to the FFT level 82 of day four of treatment. However, the graph of Figure SB shows there is a marked increase in the FFT level on the contralateral healthy limb from the baseline FFT level 80′ to the FFT level 82′ of the fourth day of treatment.

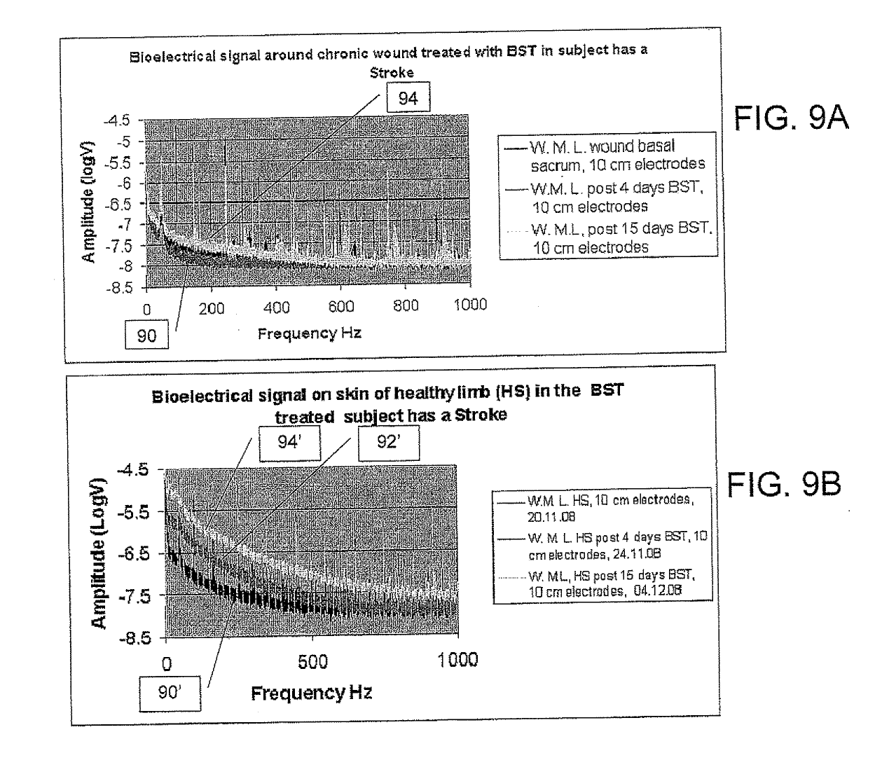

FIGS. 9A and 9B are the graphs of the FFT levels for a first patient who had suffered a Stroke and having a chronic wound. Again, the graph of FIG. 9A shows little change in the FFT levels from the baseline FFT 90 to the FFT levels 92 of day four and 94 of day fifteen of treatment. However, the graph of FIG. 9B shows there is a continued increase in the FFT level on the contralateral healthy limb from the baseline FFT level 90′ to the FFT level 92′ of the fourth day of treatment and still further increase to the FFT level 942′ of the fifteenth day of treatment.

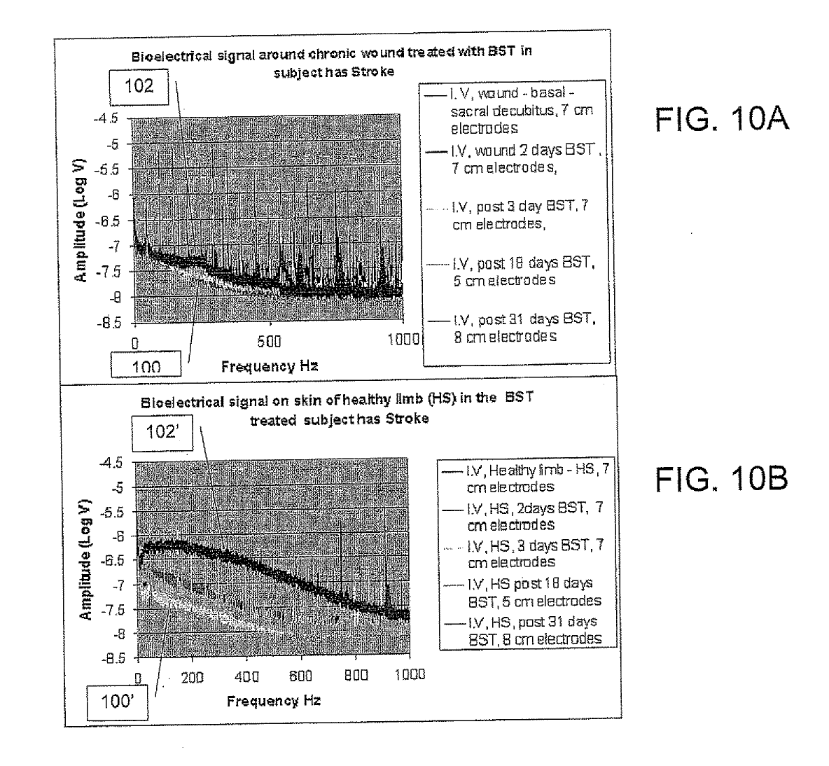

FIGS. 10A and 10B are the graphs of the FFT levels for a second patient who had suffered a Stroke and having a chronic wound. Here again, the graph of FIG. 10A shows little change in the FFT levels from the baseline FFT 100 to the FFT level 102 of day thirty-one of treatment. However, the graph of FIG. 10B shows there is a marked increase in the FFT level on the contralateral healthy limb from the baseline FFT level 100′ to the FFT level 102′ of the thirty-first day of treatment.

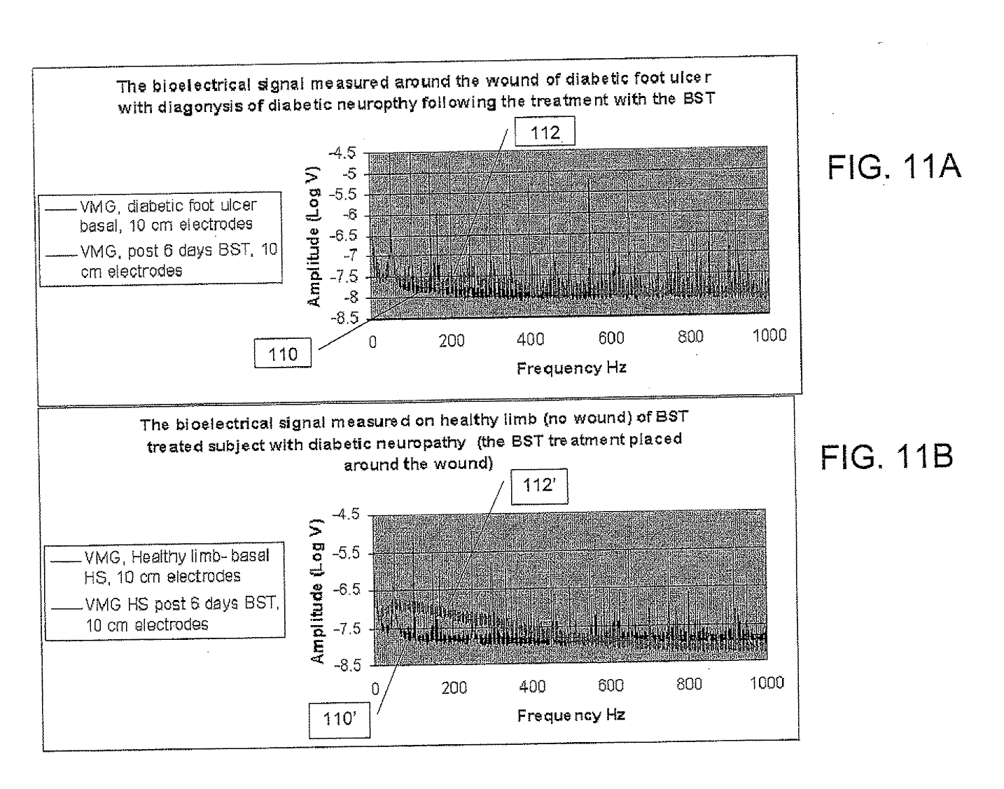

FIGS. 11A and 11B are the graphs of the FFT levels for a patient with diabetic neuropathy and having a chronic wound. Again, the graph of FIG. 10A shows little change in the FFT levels from the baseline FFT 110 to the FFT level 112 of day six of treatment. However, the graph of FIG. 11B shows there is an increase in the FFT level on the contralateral healthy limb from the baseline FFT level 110′ to the FFT level 112′ of the sixth day of treatment.

It should be noted that while the treatment durations shown above were not long enough for the healing process to begin in the wounds as indicated by the lack of change in the amplitude of the curves over the various testing sessions. However, as dramatically shown here, the endogenous bioelectrical stochastic signals generated by the human body and measured in the healthy limb, were already modulated by the application of the stochastic bioelectrical signals of the present methods.

These findings substantiate that the application of stochastic bioelectrical signals that substantially mimic the endogenous bioelectrical stochastic signals generated by the human body, such as those signals generated by the LifeWave™ BST device and described in U.S. Pat. Nos. 6,363,284, 6,393,326 and 6,941,173, will increase the amplitude of the endogenous bioelectrical stochastic signals of humans whose endogenous bioelectrical stochastic signals, as represented an FFT level measured on the skin, indicates a level of unwellness. The increase in the endogenous bioelectrical stochastic signals is represented by an increase in the amplitude of the FFT level of the patient. This is especially of interest with regard to the treatment of maladies of the CNS and dementia where an increase in endogenous bioelectrical stochastic signals from the brain may be an indication of healing.

This discovery, therefore, provides the basis for a method for modulating the amplitude of endogenous bioelectrical stochastic signals of a human. The method includes deploying at least two spaced-apart electrodes in contact with the skin surface of the patient. Then externally inducing a percutaneous flow of bioelectrical stochastic signals between the electrodes. As mentioned above, the bioelectrical stochastic signals of the present invention may be generated by a LifeWave™ BST device as a bipolar voltage wave form that substantially mimics a bipolar voltage wave four produced by a human body, as is fully described and claimed in U.S. Pat. Nos. 6,363,284, 6,393,326 and 6,941,173. It should be noted that generation of the bioelectrical stochastic signals of the present invention by a LifeWave™ BST device is intended as a non-limiting example only and that generation of such signal by any device capable of such generation is within the scope of the present invention.

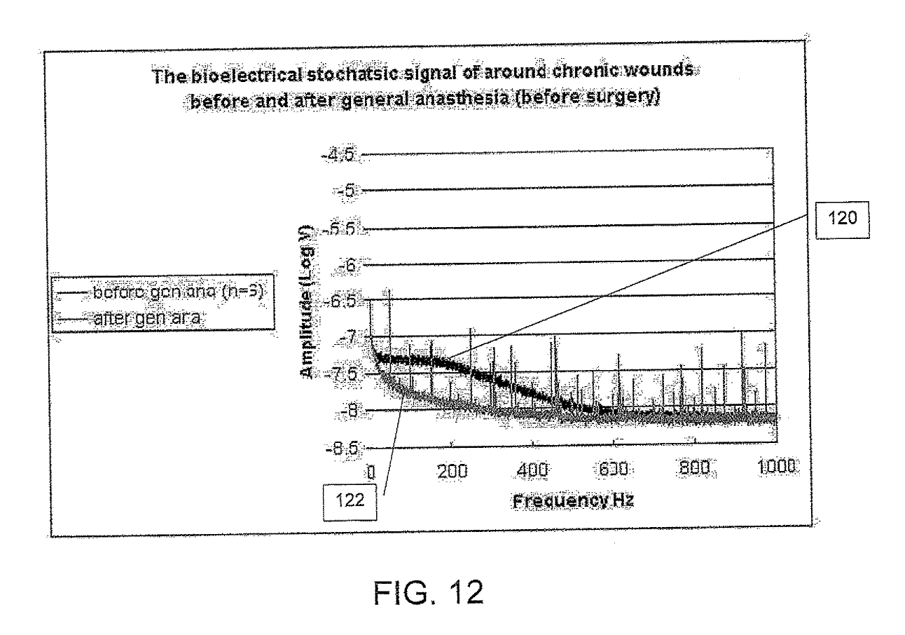

In order to better understand the ramifications of the results of their research and to further prove that the endogenous bioelectrical stochastic signals being measured originate from the brain, the present inventors collected data from patients during surgery before and after the administration of anesthesia, both general and local. The graphs of FIGS. 12-15 show their findings.

Specifically, FIG. 12 is the graphs of the FFT levels of the endogenous bioelectrical stochastic signals recorded around chronic wounds that served as the baseline data for this stage of the research. Curve 120 is the FFT levels before administration of general anesthesia and curve 122 is the FFT levels after administration of general anesthesia. It is important to note the significant drop in the signal amplitude of the FFT levels after administration of general anesthesia. This is indication that these endogenous bioelectrical signals are neuronal and derived from the brain.

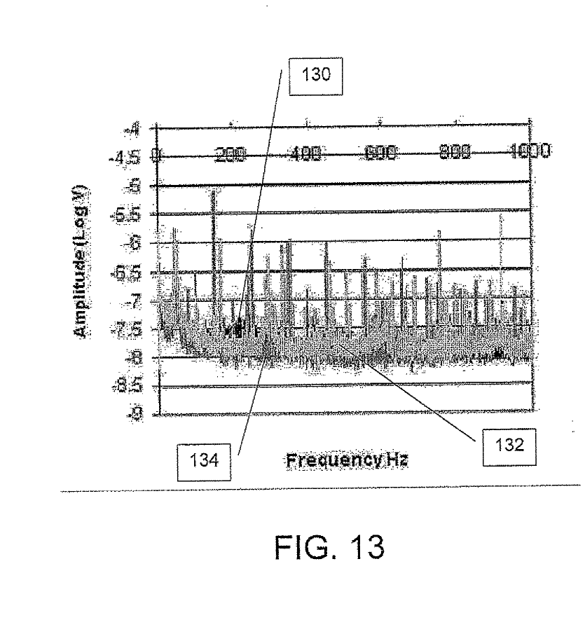

FIG. 13 is a graph of the FFT levels of the endogenous bioelectrical stochastic signals recorded on intact skin before and then during surgical incision while the patient was under general anesthesia. Curve 130 (which is mostly hidden by curves 132 and 134) is the FFT levels before administration of anesthesia, curve 132 is the FFT levels after administration of general anesthesia, and curve 134 is the FFT levels during the incision. The graph demonstrates that the FFT levels of the endogenous bioelectrical signals were not significantly changed by the surgical procedure. Therefore, these signals are neuronal (may be derived from the brain or the spinal cord) and are not affected during incision (tissue injury) when the patient is under general anesthesia.

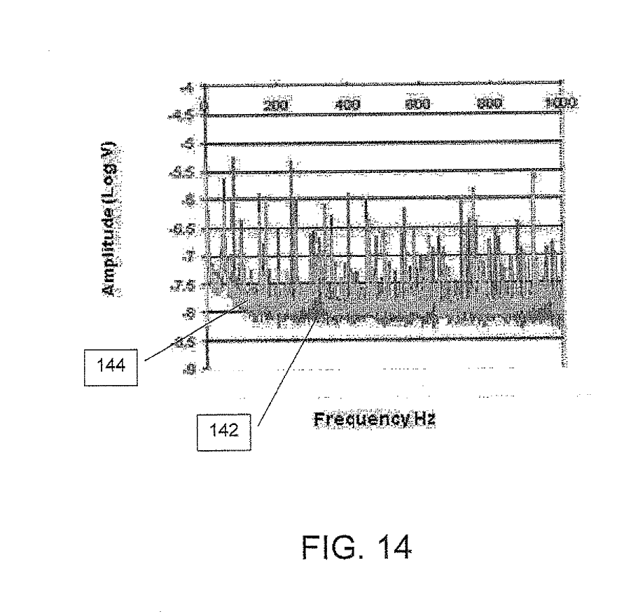

The graph of FIG. 14 shows the FFT levels of the endogenous bioelectrical signals recorded on intact skin before and during surgery incision while the patient was under spinal anesthesia. The curve of the FFT levels before administration of anesthesia is mostly hidden by curves 142 and 144. Curve 142 is the FFT levels after administration of spinal anesthesia, and curve 144 is the FFT levels during the incision. This graph also demonstrates that the FFT levels of the endogenous bioelectrical signals were not significantly changed by the surgical procedure, thereby corroborating that these signals are neuronal and are not affected during tissue injury.

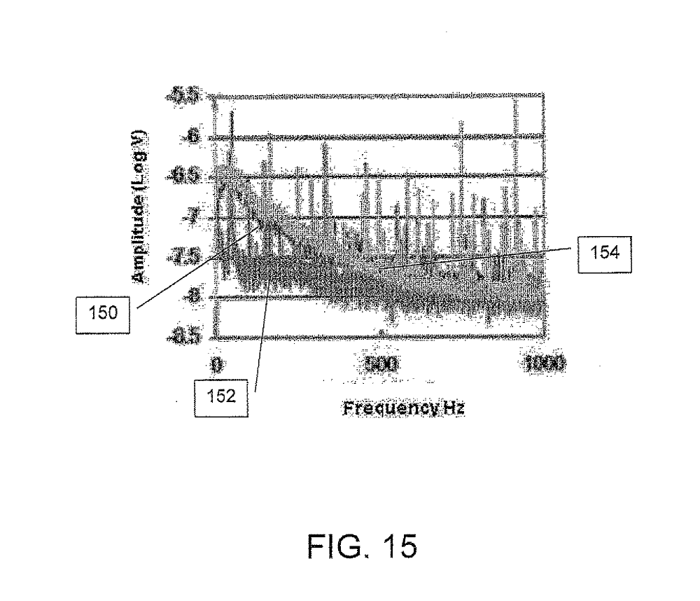

As a further measurement, the present inventors recorded the FFT levels of the endogenous bioelectrical signals recorded on intact skin before and during surgery incision while the patient was under local anesthesia. The graph of FIG. 15 is the result of those measurements. Curve 150 (which is mostly hidden by curve 154) is the FFT levels of the endogenous bioelectrical signals before administration of the local anesthesia and indicates the presents of an injury. Curve 152 is the FFT levels of the endogenous bioelectrical signals after administration of local anesthesia. The reduced FFT levels would seem to indicate a calming of the endogenous bioelectrical signals due to the anesthesia. Curve 154 is the FFT levels of the endogenous bioelectrical signals during the incision. The graph demonstrates that the FFT levels of the endogenous bioelectrical signals were significantly changed (triggered) by the surgical procedure. This graph also corroborates that these of the endogenous bioelectrical signals are neuronal signals.

It should be noted that the method of the present invention will be invaluable to medical practitioners at all levels for the diagnosis of non-visible injuries and monitoring of treatment regimens. This would be true even for first responders, who generally treat the injured with the most external bleeding first. The method of the present invention now provides the ability to identify those patients with severe non-visible injuries and treat them in a manner more suited to their injuries.

It will be appreciated that the above descriptions are intended only to serve as examples and that many other embodiments are possible within the spirit and the scope of the present invention.

What is claimed is:

1. A method for diagnosing non-visible (occult) maladies in a human patient, the method comprising:
(a) deploying at least two electrodes spaced apart on the skin of the patient;
(b) detecting and recording a bioelectrical signal in and around said electrodes;
(c) transforming said bioelectrical signal into a graph;
(d) comparing said resultant graph of the patient to at least one graph of a baseline of normal healthy humans; and
(e) determining a presents of a non-visible (occult) malady based on said comparison.

2. The method of claim 1, wherein said deploying of said electrodes in on an area of a leg of the patient.

3. The method of claim 1, wherein said detecting and recording a bioelectrical signal is implemented as detecting and recording a bioelectrical stochastic signal.

4. The method of claim 3, wherein said detecting and recording a bioelectrical stochastic signal is implemented as detecting and recording a bioelectrical neuronal signal.

5. The method of claim 3, wherein steps 1(c) and 1(d) are implemented as:
(a) transforming said stochastic signal into a voltage versus frequency spectra using a Fast Furier Transform (FFT) algorithm; and
(b) comparing a graph of a resultant FFT level of the patient to at least one graph of a baseline FFT level of normal healthy humans.

6. A method for monitoring a treatment regimen for non-visible (occult) maladies in a human patient, the method comprising:
(a) deploying at least two electrodes spaced apart on the skin of the patient;
(b) detecting and recording a first bioelectrical signal in and around said electrodes, said bioelectrical signal being a first stochastic signal;
(c) transforming said first stochastic signal into a first voltage versus frequency spectra using a Fast Furier Transform (FFT) algorithm;
(d) establishing a graph of a resultant FFT level as a baseline FFT level for the patient;
(e) administering the treatment regimen;
(f) redeploying said electrodes after a predetermined passage of time;
(g) detecting and recording at least a second bioelectrical signal in and around said electrodes, said bioelectrical signal being a second stochastic signal;
(h) transforming said second stochastic signal into a second voltage versus frequency spectra using a Fast Furier Transform (FFT) algorithm;
(i) comparing a graph of a resultant FFT level of the patient during treatment to said graph of said baseline FFT level for the patient; and
(j) determining success of the treatment regimen based on said comparison.

7. The method of claim 6, wherein steps 6(i)-6(j) are repeated according to a predetermined time table.

8. A method for modulating the amplitude of endogenous bioelectrical stochastic signals of a human, the method comprising:
(a) deploying at least two spaced-apart electrodes in contact with a skin surface of the human;
(b) externally inducing a percutaneous flow of bioelectrical stochastic signals between said electrodes;
wherein said bioelectrical stochastic signals have a bipolar voltage wave form that substantially mimics a bipolar voltage wave form produced by a human body.

9. The method of claim 8, further including increasing the amplitude of the endogenous bioelectrical stochastic signals by implementing steps 7(a) and 7(b).

10. The method of claim 8, wherein said bioelectrical stochastic signals have a bipolar voltage wave form that substantially mimics a neuronal signal produced by a human body.
